(19) United States
(12) Patent Application Publication
(10) Pub. No.: US 2012/0245414 A1
(43) Pub. Date: Sep. 27, 2012

(54) HANDLE FOR CONTROLLING INSTRUMENTS, ENDOSCOPIC INSTRUMENT COMPRISING SUCH A HANDLE, AND AN ASSEMBLY

(75) Inventor: Marcel Antonius Elisabeth Verbeek, Simpelveld (NL)

(73) Assignee: FORTIMEDIX B.V., Nuth (NL)

(21) Appl. No.: 13/069,662

(22) Filed: Mar. 23, 2011

(51) Int. Cl. A61B 1/005; A61B 1/018

(52) U.S. Cl. 600/106; 600/131

(57) ABSTRACT

A handle 1 for controlling instruments, includes a frame 2 with a center line 9, operating elements 4, 10, 24 that are connected to the frame 2, which operating elements 4, 10, 24 are movable between a first and a second position, an actuator element 6 that is controllable by the operating elements 4, 10, 24, which actuator element 6 is constructed and arranged for controlling an instrument 53 that is arranged at a distance from the handle 1. The operating elements 4, 10, 24 are arranged in a rotationally symmetric configuration that is concentric with the center line 9 of the frame 2. An endoscopic instrument 60 including a tube like element 50, a tool 53 and a handle 1, and an assembly 80 including an endoscopic instrument 60 and an endoscope 70 are also described.

### FIELD OF THE INVENTION

The present invention relates to a handle for controlling instruments. This handle comprising a frame with a center line, operating elements that are connected to the frame, which operating elements are movable between a first and a second position, an actuator element that is controllable by the operating elements, which actuator element is constructed and arranged for controlling an instrument that is arranged at a distance from the handle, wherein the operating elements are constructed and arranged for enabling a longitudinal displacement of the actuator element upon a movement of the operating elements between the first and the second position.

An instrument that can be controlled by the handle might be used in minimal invasive surgical interventions. In those interventions, the instrument is commonly used in combination with an endoscope through which the instrument is guidable into body cavities. Therefore, these instruments are commonly indicated as endoscopic instruments.

Endoscopic instruments can also be used for other than medical applications. Examples include inspection and/or repair of mechanical or electronic installations at locations that are difficult to reach. In the following description, terms as endoscopic application or endoscopic instrument might be used. However, these terms must be interpreted as covering also other applications or instruments as explained above.

The present invention further relates to an endoscopic instrument comprising a tube like element with a proximal end and a distal end, a tool and a handle according to the present invention. The handle and the tool are arranged respectively at the proximal end and the distal end of the tube like element. The tube like element and the tool are constructed and arranged for being operated by the handle according to the present invention.

The present invention also relates to an assembly of an endoscopic instrument and an endoscope that is constructed and arranged for being controlled by a handle according to the present invention.

### BACKGROUND OF THE INVENTION

Transformation of surgical interventions requiring large incisions for exposing an operation area into minimal invasive surgical interventions, i.e. requiring only small incisions for establishing access to the operation area, is a well-known and ongoing trend. To be able to perform a minimal invasive surgical intervention, a physician needs to have access to surgical instruments that enable reaching the operation area via a small incision and to remotely perform actions at the operation area. Such instruments are well-known in the art and include endoscopic instruments comprising a tube like element with a distal and proximal end, a handle connected to the proximal end and a surgical tool connected to the distal end of the tube like element. The terms distal and proximal are defined with respect to the physician that operates the instruments, i.e. the physician manipulates the proximal end of the instrument which results in an event at the distal end of the instrument at the operation area inside a patient's body. Therefore, the tube like element of the endoscopic instruments preferably is steerable as described in international patent applications WO 2009/112060 and WO 2009/127236 of the applicant, which applications were filed on 30 Jun. 2008 and 18 Apr. 2008, respectively and are here incorporated by reference.

Known handles for steerable endoscopic instruments comprise a pistol grip or pliers grip or scissors grip. Handles with a pistol grip comprise a trigger element that is constructed and arranged for activating the tool, e.g. a biopsy cutter, a pair of scissors, pliers, grippers. Steering of the tube like element of the endoscopic instrument and of the tool located at the distal end thereof is commonly done via a joystick or via a thumb controlled operating element. Known handles of the kinds mentioned above have several drawbacks that render the endoscopic instruments in which they are applied unsuitable for being used in minimal invasive interventions.

A first drawback of the abovementioned known handles is that although an operator, e.g. a physician, can of course learn how to steer the endoscopic instrument and the tool at the distal end thereof with the thumb, this is not a natural mechanism of action and reaction and usually does not give a one on one deflection feedback. In addition, operating a trigger element, for example for closing a pair of scissors or pliers, is not a natural mechanism of action and reaction and usually does not provide logical force feedback.

A second drawback is that rotating the endoscopic instrument requires not only rotation of a wrist or rotation of the wrist and under arm but in some cases also rotation of a whole arm or even rotation or repositioning of substantially the whole body of the operator.

A third drawback is that the handles described above can be quite voluminous. If in a minimal invasive intervention two endoscopic instruments comprising such handles are to be used side by side, it might at least be difficult if not impossible to do so as the handles could be in each other's way. As a result, freedom of manipulation would at least severely be reduced. To solve this, in practice two incisions at sufficient distance from each other would be necessary.

A fourth drawback is that the known handles commonly comprise many separate parts and pulling cable mechanisms, which are complex to assemble and therefore expensive to manufacture. As a result, these kinds of handles are commonly applied in instruments that are reused because of the relatively high costs involved. Reuse of surgical instruments might imply risks with respect to cleaning and sterilization and require proper equipment and manpower in the hospital. Unfortunately, infections as a result of surgical instruments that have not been cleaned and sterilized properly frequently occur.

### SUMMARY OF THE INVENTION

It is an object of the present invention to provide a handle for controlling instruments, such as endoscopic instruments, that preempts or at least reduces the drawbacks of the abovementioned known handles. It is also an object of the present invention to provide an endoscopic instrument that is controllable by the handle according to the present invention. It is a further object of the present invention to provide an assembly of an endoscopic instrument and an endoscope that is constructed and arranged for being controlled by a handle according to the present invention.

At least one of these objects is achieved by a handle according to the present invention, wherein the operating elements are arranged in a rotationally symmetric configuration that is concentric with the center line of the frame, wherein the operating elements are movable between the first and second positions by a displacement in a direction substantially perpendicular to the center line of the frame. The handle according to the present invention has a rotationally symmetric outer circumference when the operating elements are in or in between the first and second positions. Such a rod like handle provides a pen-like grip that enables convenient and natural manipulation of the handle between thumb and fingers. Therefore, the handle should have an outer diameter in a radial direction with respect to the center line of the frame that lies in a range from about 10-50 millimeters, preferably in a range from about 20-40 millimeters.

By interconnecting the operating elements via a resilient element, such as a rubber ring or rubber cover, all operating elements are movable substantially simultaneously between the first and second position when at least one of them is displaced in a direction substantially perpendicular to the center line of the handle. As a result, by simply squeezing the handle anywhere at its outer circumference between thumb and fingers, an actuating element that is constructed and arranged for controlling an event at a distal end of an endoscopic instrument is controlled such that a tool at a distal end of an endoscopic instrument changes state, e.g. is activated or deactivated. Upon releasing the handle, the operating elements are moved from the second to the first position as they are prestressed towards the first position by a first pretensioning element. As a result, the actuating element is controlled such that the tool at the distal end of the endoscopic instrument changes state, e.g. is deactivated or activated.

The outer circumference of the handle according to the present invention remains rotationally symmetric when the operating elements are moved between the first and second positions. As a result, manipulation of the handle remains convenient and controllable.

Rotation of the endoscopic instrument over the complete 360° can easily be done by rotating the handle between thumb and fingers, without requiring any adjustment in position of the arms and/or the whole body of the operator.

Steering of the endoscopic instrument can be achieved by moving the handle in any desired direction. Steering can be done in situations in which the handle is un-squeezed, i.e. operating elements are in the first position, or squeezed, i.e. operating elements are in between the first and the second position or in the second position, without requiring rotation of the whole arm or even rotation or repositioning of substantially the whole body of the operator. The handle according to the present invention enables steering of the endoscopic instrument and controlling of the tool located at the distal end thereof by simple manipulations performed by the thumb and fingers and/or the wrist and/or under arm of the operator. As a result, the handle according to the present invention provides a natural mechanism of action and reaction and a one on one deflection feedback. In addition, the handle according to the present invention provides logical force feedback. The force feedback can be one on one (1:1) but it can also be tuned by applying a second pretensioning element that prestresses the operating elements towards the second position. Because of the clear feedback the operator, e.g. a physician, can clearly feel which forces are exerted by the endoscopic instrument and/or the tool at the distal end thereof on treated or surrounding tissue.

The slim and compact pen-like construction of the handle according to the present invention might enable the use of two endoscopic instruments comprising such handles side by side in a minimal invasive intervention. As the freedom of manipulation might significantly be increased, it might no longer be necessary to make two incisions at sufficient distance from each other in certain applications. Instead a single incision might be made and the two instruments might be guided through a single endoscope.

According to another aspect of the present invention an endoscopic instrument is provided comprising a tube like element with a proximal end and a distal end, a tool and a handle according to any of the preceding claims, wherein the handle and the tool are arranged respectively at the proximal end and the distal end of the tube like element, wherein the tube like element and the tool are constructed and arranged for being operated by the handle.

The handle is constructed and arranged for controlling the tool located at the distal end of the endoscopic instrument by mechanical means like a pulling wire. Examples include closing and opening of cutting blades of a pair of surgical scissors or inserting or retracting a needle. The handle can also be constructed and arranged for controlling such tools by means of making and/or breaking an electrical contact and/or by means of opening and/or closing a pneumatic or hydraulic valve or combinations of all means mentioned. The handle according to the present invention is also constructed and arranged for rotating the endoscopic instrument and steering of the distal end thereof.

The actual steering mechanism of the endoscopic instrument is located in the tube like element. The proximal and distal ends thereof comprise flexible portions that are constructed and arranged for steering the tube like element of the endoscopic instrument by moving the handle in any direction. The tube like element comprises longitudinal elements that are constructed and arranged for transferring a displacement of the proximal end to the distal end resulting in a change in orientation thereof. The distal end comprises at least two independent flexible portions. This enables for example making S-like curves with the distal end of the tube like element.

The proximal and distal ends comprise a corresponding number of flexible portions, wherein each flexible portion at the proximal end is connected by means of its own set of longitudinal elements to a flexible portion at the distal end. A detailed description of such steerable tube like elements and preferred processes for producing them have been described in international patent applications WO 2009/112060 and WO 2009/127236 of the applicant, which applications were filed on 30 Jun. 2008 and 18 Apr. 2008, respectively and are here incorporated by reference. These steerable tube like elements provide enhanced guiding capabilities of the endoscopic instrument and enhanced positioning capabilities of a tool, e.g. a surgical pair of scissors, a biopsy cutter, pliers or grippers, at an operation location.

According to another aspect of the present invention an assembly is provided comprising an endoscopic instrument and an endoscope that is constructed and arranged for being controlled by a handle according to the present invention. Therefore, the endoscope comprises a tube like element having proximal and distal ends that comprise flexible portions that are constructed and arranged for steering the endoscope by moving the handle according to the present invention in any direction.

### BRIEF DESCRIPTION OF THE DRAWINGS

These and other features and effects of the present invention will be explained in more detail below with reference to drawings in which preferred and illustrative embodiments of the invention are shown. The person skilled in the art will realize that other alternatives and equivalent embodiments of the invention can be conceived and reduced to practice without departing from the scope of the present invention.

FIG. 1 shows a schematic representation of a longitudinal cross section of a conventional handle, wherein the operating element is a single lever in the first position.

FIG. 1a shows a schematic representation of a longitudinal cross section of the handle according to FIG. 1, wherein the single lever is in the second position.

FIG. 2 shows a schematic representation of a longitudinal cross section of a handle according to the present invention, wherein a first group of levers are arranged in a rotationally symmetric configuration that is concentric with the center line of the frame.

FIG. 2a shows a cross section of the handle in FIG. 2 in direction IIa.

FIG. 3 shows a schematic representation of a longitudinal cross section of a preferred embodiment of the handle according to the present invention, wherein a first and a second group of levers are arranged in a rotationally symmetric configuration that is concentric with the center line of the frame. The first group of levers is arranged mirror wise with respect to a second group of levers. The levers of both the first and the second groups are in the first position.

FIG. 3a shows a schematic representation of a longitudinal cross section of the handle according to FIG. 3, wherein the levers of the first and second groups are in the second position.

FIG. 4 shows a longitudinal cross section of the preferred embodiment of the handle according to the present invention, wherein the levers are in the first position.

FIG. 4a shows a cross section of the handle in FIG. 4 along line IVa-IVa.

FIG. 4b shows a longitudinal cross section of the preferred embodiment of the handle according to the present invention, wherein the levers are in the second position.

FIG. 5 shows a schematic representation of a longitudinal cross section of an embodiment of the handle according to the present invention, wherein the handle comprises conical sliding planes along which the operating elements can be displaced. The operating elements are in the first position.

FIG. 5a shows a schematic representation of a longitudinal cross section of the embodiment of the handle according to FIG. 5, wherein the operating elements are in the second position.

FIG. 6 shows a schematic representation of a longitudinal cross section of an embodiment of the handle according to the present invention, wherein the handle comprises a frame having an interior volume that is in communicating relationship with an interior volume of a housing. The interior volumes of the frame and housing can be filled with a fluid, e.g. a liquid or a gas. The operating elements are in the first position.

FIG. 6a shows a schematic representation of a longitudinal cross section of the embodiment of the handle according to FIG. 6, wherein the operating elements are in the second position.

FIG. 7 shows a schematic representation of a longitudinal cross section of an embodiment of the handle according to the present invention, wherein the operating elements comprise first and second arms that are hingeably connected. The operating elements are in the first position.

FIG. 7a shows a schematic representation of a longitudinal cross section of the embodiment of the handle according to FIG. 7, wherein the operating elements are in the second position.

FIG. 8 shows a side view of an endoscopic instrument comprising a handle according to the present invention, a tube like element and a surgical pair of scissors, wherein the handle is located at the proximal end of the tube like element and the surgical pair of scissors is located at the distal end of the tube like element.

FIG. 8a shows a side view of the endoscopic instrument according to FIG. 8, wherein the distal end of the tube like element and the surgical pair of scissors that is attached to it are deflected downward with respect to their positions in FIG. 8 as a result of a displacement of the handle at the proximal end of the tube like element in a direction upward with respect to its position in FIG. 8.

FIG. 8b shows a side view of the endoscopic instrument according to FIG. 8, wherein the surgical pair of scissors is closed as a result of squeezing of the handle, i.e. displacing the operating elements of the handle from the first to the second position.

FIG. 9 shows a side view of an assembly comprising two endoscopic instruments that are guided through a single endoscope. The endoscopic instruments and the endoscope comprise a handle according to the present invention and they are constructed and arranged for being controlled by that handle.

The figures are not necessarily drawn to scale. In the figures identical components are denoted by the same reference numerals.

### DETAILED DESCRIPTION

The handle as shown in the annexed drawings can be used with endoscopic instruments for medical applications but its use is not restricted to that. It may also be used in other applications such as technical applications in which endoscopic instruments are used for handling or viewing parts of machines or installations which are otherwise difficult to reach. The handle according to the present invention as used in this description will implicitly include these applications.

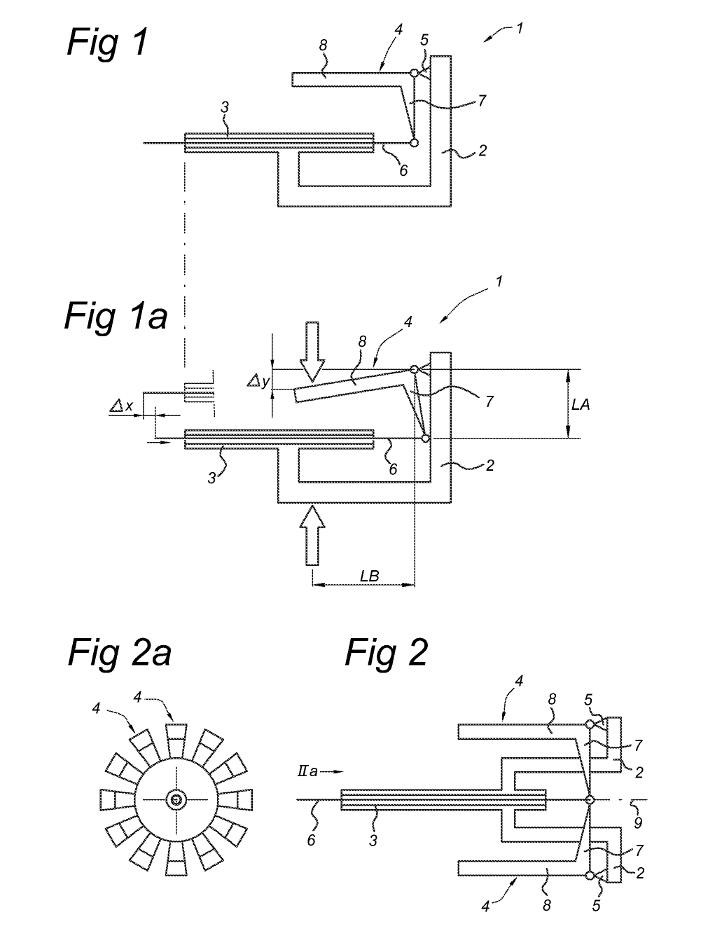

FIG. 1 shows a schematic representation of a longitudinal cross section of a known handle 1, wherein the operating element 4 is a single lever in the first position. Handle 1 comprises a frame 2 and a housing 3 that are connected such that their positions remain stationary with respect to each other when lever 4 is moved from the first to the second position by pivoting around pivot point 5. An actuator element 6, which in this embodiment is a pulling wire, can be displaced with respect to housing 3 when the lever 4 pivots around pivot point 5. A longitudinal displacement of pulling wire 6 results in controlling a tool located at a distal end of an endoscopic instrument. Examples include closing and opening of cutting blades of a pair of surgical scissors and/or inserting or retracting a needle, etc.

FIG. 1a shows a schematic representation of a longitudinal cross section of the handle 1 according to FIG. 1, wherein the single lever 4 is in the second position. Comparison of FIGS. 1 and 1a shows that the pulling wire is displaced an amount Δx due to a displacement Δy of lever 4. FIG. 1a shows that lever 4 comprises first arms 7 and second arms 8. First arms 7 are positioned substantially radially with respect to housing 3 and have a length LA. Second arms 8 are arranged substantially parallel to housing 3 and have a length LB. In order to achieve a more powerful displacement of pulling wire 6 it is clear that length LA has to be made smaller with respect to length LB. A drawback of this approach is that displacement Δx decreases when length LA is further reduced with respect to length LB. This can be solved by increasing the dimensions of handle 1 while keeping the ratio of length LA and length LB constant. However, in practice this solution is only of limited value as the outer diameter of handle 1 cannot be increased beyond a value that makes handle 1 impractical for manipulations between thumb and fingers of an operator. Therefore, the outer diameter in a radial direction with respect to housing 3 of handle 1 should be in a range from about 10-50 millimeters, preferably in a range from about 20-40 millimeters.

FIG. 2 shows a schematic representation of a longitudinal cross section of a handle 1 according to the present invention, wherein a first group of levers 4 are arranged in a rotationally symmetric configuration that is concentric with the center line 9 of the frame 2. This configuration enables a manipulation of the levers 4 irrespective of a rotational position of handle 1. FIG. 2 also shows that frame 2 and housing 3 are connected such that their positions remain stationary with respect to each other when levers 4 are moved from the first to the second position by pivoting around pivot points 5.

FIG. 2a shows a cross section of the handle in FIG. 2 along line IIa-IIa. It clearly shows that the first group of levers 4 is arranged in a rotationally symmetric configuration that is concentric with the center line 9 of the frame 2.

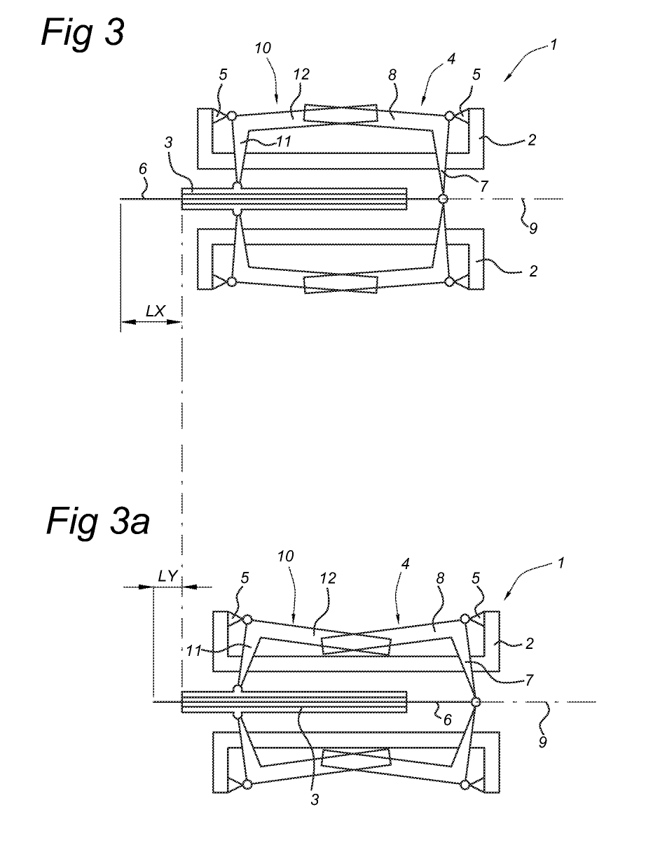

FIG. 3 shows a schematic representation of a longitudinal cross section of a preferred embodiment of the handle 1 according to the present invention, wherein a first group of levers 4 and a second group of levers 10 are arranged in a rotationally symmetric configuration that is concentric with the center line 9 of the frame 2. The first group of levers 4 is arranged mirror wise with respect to a second group of levers 10. The levers 4, 10 of both the first and the second groups are in the first position.

The preferred embodiment of the handle 1 according to the present invention should achieve a larger displacement Δx of pulling wire 6 while maintaining the ratio of length LA and length LB for achieving a sufficient displacement force.

FIG. 3 shows that the levers 4 of the first group and the levers 10 of the second groups are arranged in an interdigitated configuration. This enables a simultaneous displacement of levers 4 of the first group and levers 10 of the second group when the handle 1 is squeezed between thumb and fingers. It will be clear to the person skilled in the art that many interdigitated configurations of the levers of the first and second groups can be envisaged. It is possible that the levers of the first group are divided into pairs by the levers of the second group. It is also possible that the levers of the first group are divided into pairs by pairs of levers of the second group. These examples show that the number of levers in the first and second groups can be different.

FIG. 3 also shows that the frame 2 and housing 3 are arranged such that they are movable with respect to each other when the levers 4, 10 are moved from the first to the second position. This arrangement of the frame 2 with respect to the housing 3 yields an additional displacement of the pulling wire 6 compared to a handle 1 comprising only the first group of levers 4. In addition, FIG. 3 shows that each of the first and second groups of levers 4, 10 comprises at least one lever.

FIG. 3a shows a schematic representation of a longitudinal cross section of the handle according to FIG. 3, wherein levers 4 of the first group and levers 10 of the second group are in the second position. From FIGS. 3 and 3a, it can be seen that the displacement of pulling wire 6 is defined as the difference between length Lx and length Ly, i.e. Lx−Ly. It has to be noted that the displacement of pulling wire 6 is almost doubled with respect to a handle 1 only comprising the first group of levers 4. As a result, handle 1 according to the preferred embodiment achieves a significant increase in the amount of displacement of pulling wire 6 while maintaining the ratio of length LA and length LB and the desired puling force.

It has to be noted that in the case of applications in which the pulling wire 6 only has to be displaced about an amount Δx a first group of levers 4 suffices. Furthermore, in those cases in which no actuator element 6 is required for controlling a tool that is located at a distal end of an endoscopic instrument the same arrangements of levers can be used, i.e. only a first group of levers 4 that are arranged in a rotationally symmetric configuration that is concentric with the center line 9 of the frame 2 or a first group of levers 4 and a second group of levers 10 that are arranged in a rotationally symmetric configuration that is concentric with the center line 9 of the frame 2, wherein the first group of levers 4 is arranged mirror wise with respect to a second group of levers 10. Using one of these arrangements, it is possible for example to make or break an electrical contact and/or to control a potentiometer and/or to open and close a valve in a dosed manner.

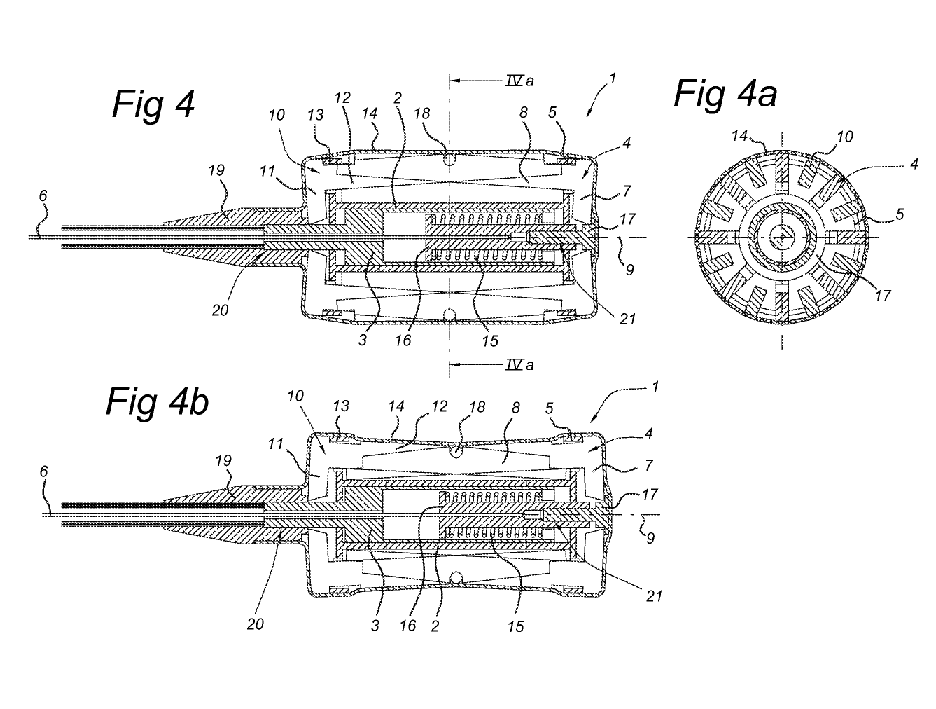

FIG. 4 shows a longitudinal cross section of the preferred embodiment of the handle 1 according to the present invention, wherein the levers 4, 10 are in the first position.

FIG. 4a shows a cross section of the handle in FIG. 4 along line Ia-Ia. It can be seen that the first group of levers 4 and the second group of levers 10 are arranged in an interdigitated configuration. As shown in FIG. 4a, the first and second groups of levers 4, 10 comprise eight levers each. The levers 4, 10 are pivotable around pivot points 5 between the first and second position. The pivot points 5 are arranged in a rotationally symmetric configuration that is concentric with the center line 9 of the frame 2. The pivot points 5 do not need to be fixedly attached to the frame 2 or levers 4, 10. As shown in FIG. 4a, a circular element, which rests on the levers 4, 10, can be used to hold levers 4,10 in place and provide the pivot points 5. An effect of implementing the pivot points 5, 13 as is shown in FIGS. 4-4b is that production costs can significantly be reduced as less parts are required and assembly is easier and quicker.

FIGS. 4-4b show that the levers 4, 10 of the first and second groups provide handle 1 with a rotationally symmetric outer circumference when the levers 4, 10 are in the first and second positions. FIG. 4a clearly shows that the outer circumference of handle 1 in a radial direction with respect to the center line 9 of handle 1 is substantially circular.

Especially for medical applications, it is required that handle 1 is hermetically sealed from an outside environment. This can be done by a sealing 14 that fully encloses the outer circumference of handle 1 as is shown in FIGS. 4 and 4b. An effect of using a resilient sealing 14 is that it can provide a pretension force that prestresses the levers 4, 10 towards the second position. Suitable resilient materials are highly stretchable and comprise for example a membrane comprising silicone or latex.

From FIG. 4 it is clear that resilient membrane 14, in addition to resilient element 18, will force all levers 4, 10 from the first to the second position if only one or a few of them are manipulated between thumb and fingers of the operator.

In order to maintain levers 4, 10 in the first position, handle 1 comprises a first pretensioning element 15 that is constructed and arranged for prestressing the levers 4, 10 towards the first position. In the preferred embodiment shown in FIGS. 4-4b, the first pretensioning element 15 is a spring. Spring 15 is arranged inside housing 3 and around plunger element 16. Housing 3 is arranged such that frame 2 is movable with respect to it when the levers 4, 10 are moved between the first and second positions. Spring 15 forces plunger element 16 and housing 3 in opposite longitudinal directions to maintain the first position of the levers 4, 10. Plunger element 16 is connected via connecting element 17 with the levers 4 of the first group. Housing 3 is connected via connecting element 19 with the levers 10 of the second group.

FIG. 4b shows a longitudinal cross section of the preferred embodiment of the handle 1 according to the present invention, wherein the levers 4, 10 are in the second position. As stated above, the levers 4, 10 can be prestressed towards the second position by resilient sealing 14. However, prestressing of levers 4, 10 towards the second position can also be achieved or, if necessary, can be enhanced by a second pretensioning element 18 that is arranged around the levers 4, 10 in a radial direction with respect to the center line 9 of frame 2. The second pretensioning element 18 can be a resilient circular element. Pretensioning element 18 can comprise rubber or a metal spring.

When the levers 4, 10 are moved from the first to the second position, first lever arms 7 of levers 4 displace connecting element 17 in a longitudinal direction opposite to that in which plunger element 16 is forced by spring 15. At the same time, frame 2 is longitudinally displaced in the direction of first arms 7 of levers 4 by first arms 11 of levers 10 via connecting element 19.

FIGS. 4 and 4b show that handle 1 has a rotationally symmetric outer circumference when the levers 4, 10 are in the first and second positions and any position in between. It will be clear to the person skilled in the art that the first position is only maintained if the force exerted by spring 15 results in a larger and oppositely directed torque than the torque resulting from the force exerted by resilient sealing 14 and/or the second pretensioning element 18. These torques are tunable such that an operating force is established that allows force feedback from the activated instrument resulting from manipulation or cutting the treated tissue.

With the preferred embodiment as shown in FIGS. 4-4b it is aimed to provide a handle 1 that can be assembled as simply as possible using a minimal amount of parts with a simple construction and manufacturability. By also reducing the costs of the materials used, a handle 1 can be provided that can be disposed after use. By providing a disposable handle 1 the risks with respect to cleaning and sterilization can be eliminated.

Materials that can be used in the production process are thermoplastic materials that might be fiber reinforced and are suitable for injection molding. Examples of such materials are nylon, polyvinyl chloride (PVC), acrylonitrile butadiene styrene (ABS), polyethylene terephthalate (PET) and others as long as they are sufficiently strong and rigid from a mechanical point of view. Other preferable materials are plastics, metals or resins that can be shaped with for example three dimensional printing. With respect to metals, other suitable choices are alloys that can be injection molded and/or alloys that are easily machinable.

As can be seen from FIGS. 4-4b, the preferred embodiment of handle 1 comprises parts, e.g. frame 2, housing 3, plunger 16 that are slideable into each other. The preferred embodiment of handle 1 comprises only two screw connections, i.e. a screw connection 20 between housing 3 and connecting element 19 and a screw connection 21 between plunger 16 and connecting element 17. The screw connections 20, 21 are also used to adjust, during assembly, the first position of the handles 4, 10. These two screw connections 20, 21, the shape of the parts and the resilient sealing 14 keep together all parts of handle 1. All other connections comprise loose bearings, such as element 5, and/or fitting connections.

An embodiment of handle 1 could be assembled from parts that are cheaply connectable without needing any crew connections at all. Although adjustment of the first position of the handles 4, 10 might not be possible, such an embodiment could reduce the costs of handle 1.

An embodiment of handle 1 could comprise real hinge constructions that are fixed to the frame 2 and levers 4, 10. The costs of this embodiment would be higher as more parts are required and assembly is more difficult.

An embodiment of handle 1 could comprise levers 4,10 that are integrated with connecting elements 17, 19 to form a single integrated part. This could possibly further reduce the costs of handle 1.

It will be clear to the person skilled in the art that many alternative embodiments are possible without departing from the scope of the present invention.

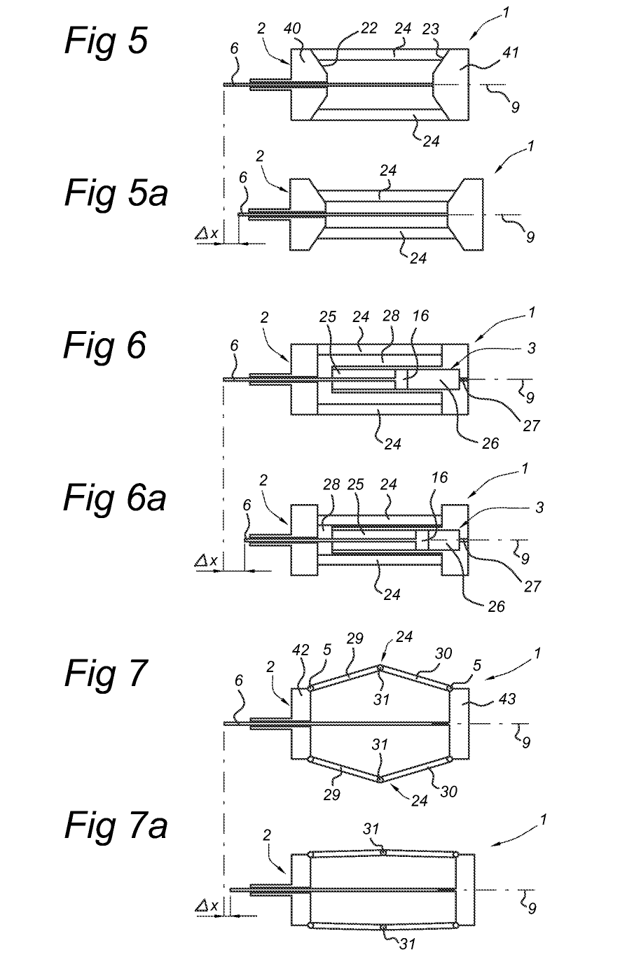

FIG. 5 shows a schematic representation of a longitudinal cross section of an embodiment of the handle 1 according to the present invention, wherein the frame 2 of the handle 1 comprises two parts 40, 41 comprising conical sliding planes 22, 23 respectively. The two parts 40, 41 of the frame 2 are connected via operating elements 24. When moved between the first and second positions, the operating elements 24 can be guided along the conical sliding planes 22, 23. When the operating members 24 are moved from the first to the second position the length of the handle 1 will increase in longitudinal direction as parts 40 and 41 are displaced in a direction opposite to each other.

The main difference between the handles shown in FIGS. 4 and 5 is in the implementation of the mechanism that enables movement of the operating elements 24 between the first and second positions. The operating elements 24, which are also arranged in a rotationally symmetric configuration that is concentric with the center line 9 of the frame 2, can be manipulated with thumb and fingers by an operator. By squeezing the handle 1 the operating elements 24 are displaced in a direction substantially perpendicular to the center line 9 of the frame 2. This radial displacement is transferred into a longitudinal displacement Δx of the actuator element 6, which for example is a pulling wire.

The first position of the operating elements 24 is maintained by a first pretensioning element. A second pretensioning element can be arranged around the operating elements 24 in a radial direction with respect to the center line 9 of the frame 2 of the handle 1 for prestressing the operating elements 24 towards the second position. A resilient sealing will also be arranged around the handle 1 in order to hermetically seal it from an environment.

The embodiment of the handle 1 shown in FIGS. 5 and 5a does not comprise pivot points. It also does not necessarily comprise a housing, a plunger element and connecting elements. Therefore, it is a very simple and low cost implementation of the handle 1 according to the present invention. The embodiment according to FIGS. 4-4b is however preferred as a more controllable and/or more powerful longitudinal displacement of the actuator element 6 can possibly be achieved.

FIG. 6 shows a schematic representation of a longitudinal cross section of an embodiment of the handle 1 according to the present invention, wherein the handle 1 comprises a frame 2 having an interior volume 28 that is in communicating relationship with an interior volume 25 of a housing 3. The interior volumes 28 and 25 can be filled with a fluid, e.g. a liquid or a gas. The operating elements 24 are prestressed towards the first position by a first pretensioning element like a spring like element that is arranged in interior volume 26 and/or by an increased pressure of a gas that is contained in interior volume 26 and/or by a spring like element that is arranged in frame 2. The latter embodiment of the first pretensioning element would be most suitable when interior volumes 28 and 25 are filled with a gas. The former two embodiments of the first pretensioning element would be most suitable when interior volumes 28 and 25 are filled with a liquid.

Again the main difference between the handles shown in FIGS. 4 and 5 and FIG. 6 is in the implementation of the mechanism that enables movement of the operating elements 24 between the first and second positions. The operating elements 24, which also in the case of the embodiment of the handle 1 shown in FIG. 6 are arranged in a rotationally symmetric configuration that is concentric with the center line 9 of the frame 2, can be manipulated with thumb and fingers by an operator. A second pretensioning element can be arranged around the operating elements 24 in a radial direction with respect to the center line 9 of the frame 2 of the handle 1 for prestressing the operating elements 24 towards the second position. A resilient sealing will also be arranged around the handle 1 in order to hermetically seal it from an environment.

Frame 2 comprises an interior volume 28 that is in a communicating relationship with an interior volume of housing 3. The interior volume of housing 3 is divided into two parts 25, 26 by plunger 16. Squeezing the handle 1 results in a displacement of the operating elements 24 in a direction substantially perpendicular to the center line 9 of the frame 2 towards the second position. As a result, the interior volume 28 of frame 2 decreases. This decrease is compensated by an increase in volume of part 25 and a decrease in volume 26 of the interior volume of the housing 3. The volume of part 26 can be decreased because of the communicating connection 27 between part 26 and an environment outside the handle 1 or by filling this volume with a compressible gas. Due to the increase of the volume of part 25 of the interior volume of the housing 3 plunger element 16 is displaced in a longitudinal direction. As the plunger 16 is connected to an actuator element 6, e.g. a pulling wire, the radial displacement of operating elements 24 is transferred into a longitudinal displacement Δx of the actuator element 6.

If no force is applied to the handle 1, the operating elements 24 are prestressed towards the first position by the first pretensioning element. As a result the volume 28 of the frame 2 is increased. The increase in volume 28 is compensated by a decrease of the volume of part 25 and an increase in the volume of part 26 of the housing 3. The volume of part 26 of the housing 3 can increase because of the communicating connection 27 between part 26 of the interior volume of the housing 3 and the outside environment of the handle 1. As a result of the changing volumes of parts 25 and 26 of the housing 3, plunger element 16 is displaced in an opposite longitudinal direction with respect to the transition of the operating elements 24 from the first to the second position. This situation is most likely for an embodiment of handle 1 in which interior volumes 28 and 25 are filled with a gas.

In an embodiment of handle 1 in which interior volumes 28 and 25 are filled with a liquid, the plunger 16 is prestressed towards the first position by the first pretensioning element which is for example a spring or a compressed gas arranged in volume 26. If no force is applied to the handle 1, volume 25 is decreased. The decrease in volume 25 is compensated by an increase of volume 28. As a result of the increasing volume 28, operating elements 24 will move and will be kept in the first position.

FIG. 7 shows a schematic representation of a longitudinal cross section of an embodiment of the handle 1 according to the present invention, wherein the operating elements comprise first and second arms that are hingeably connected. The operating elements are in the first position.

The main difference between the embodiments of the handle 1 shown in FIGS. 4 and 7 is that the embodiment shown in FIG. 7 does not comprise a housing inside the frame and that the operating elements 24 comprise first arms 29 and second arms 30 that are hingeably connected by hinge point 31.

The frame 2 of the handle 1 comprises two parts 42, 43 that are connected via operating elements 24. When moved between the first and second positions, the operating elements 24 pivot around pivot points 5 that are arranged on the parts 42, 43 of the frame 2 respectively. When the operating members 24 are moved from the first to the second position the length of the handle 1 will increase in longitudinal direction as parts 42 and 43 are displaced in a direction opposite to each other.

As the embodiment of the handle 1 shown in FIG. 7 comprises less parts than the preferred embodiment shown in FIG. 4, it is a simpler and possibly cheaper implementation of the handle 1 according to the present invention. The embodiment according to FIGS. 4-4b is however preferred as a more controllable and more powerful longitudinal displacement of the actuator element 6 can possibly be achieved.

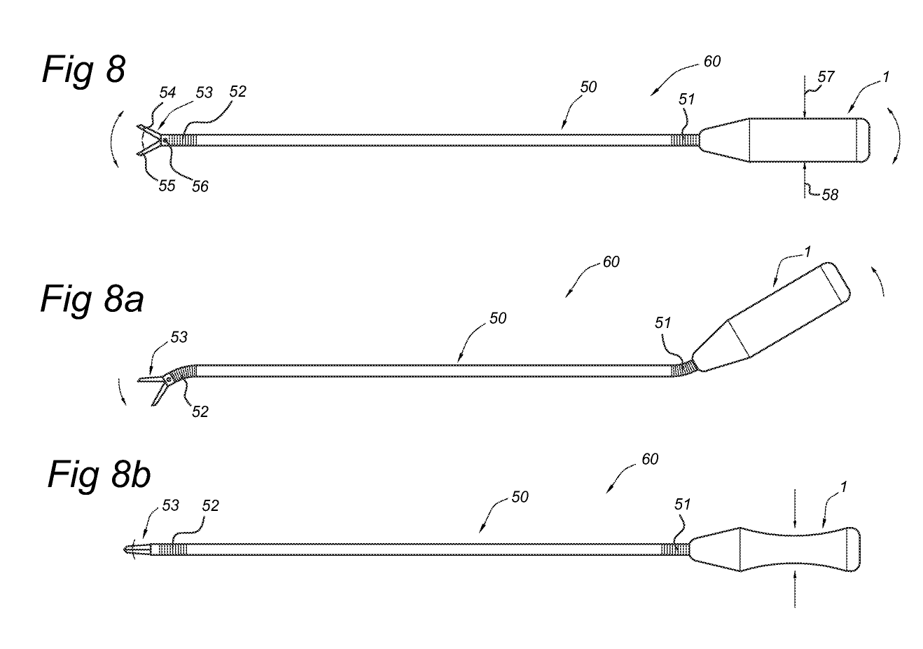

FIG. 8 shows a side view of an endoscopic instrument 60 comprising a handle 1 according to the present invention, a tube like element 50 and a surgical pair of scissors 53. The handle 1 is located at the proximal end 51 of the tube like element 50 and the surgical pair of scissors 53 is located at the distal end 52 of the tube like element 50. The handle 1 of the endoscopic instrument 60 as shown in FIG. 8 is in the first position. The surgical pair of scissors 53 is open, i.e. cutting blades 54, 55 are substantially in contact with each other at hinge connection 56. Arrows 57, 58 indicate the direction in which the handle 1 can be manipulated by an operator, e.g. a physician, to operate the surgical pair of scissors 53.

The actual steering mechanism of the endoscopic instrument 60 is located outside the handle 1 in the tube like element 50. FIG. 8 schematically shows that the proximal 51 and distal 52 ends of the tube like element 50 comprise flexible portions that are constructed and arranged for steering the tube like element 50 of the endoscopic instrument 60 by moving the handle 1 in any direction. Such a steerable tube like element 50, which has been described in international patent applications WO 2009/112060 and WO 2009/127236 of the applicant, which applications were filed on 30 Jun. 2008 and 18 Apr. 2008, respectively and are here incorporated by reference as has already been mentioned above, provides enhanced guiding capabilities of the endoscopic instrument 60 and enhanced positioning capabilities of the surgical pair of scissors 53 at an operation location.

FIG. 8a shows a side view of the endoscopic instrument 60 according to FIG. 8, wherein the distal end 52 of the tube like element 50 and the surgical pair of scissors 53 that is attached to it are deflected downward with respect to their positions in FIG. 8 as a result of a displacement of the handle 1 at the proximal end 51 of the tube like element 50 in a direction upward with respect to its position in FIG. 8.

FIG. 8b shows a side view of the endoscopic instrument 60 according to FIG. 8, wherein the surgical pair of scissors 53 is closed, i.e. the cutting blades 54, 55 are in contact over their longitudinal length, as a result of squeezing of the handle 1, i.e. displacing the operating elements of the handle 1 from the first to the second position. FIGS. 8-8b clearly show that the outer circumference of the handle 1 is rotationally symmetric in both the first and the second position.

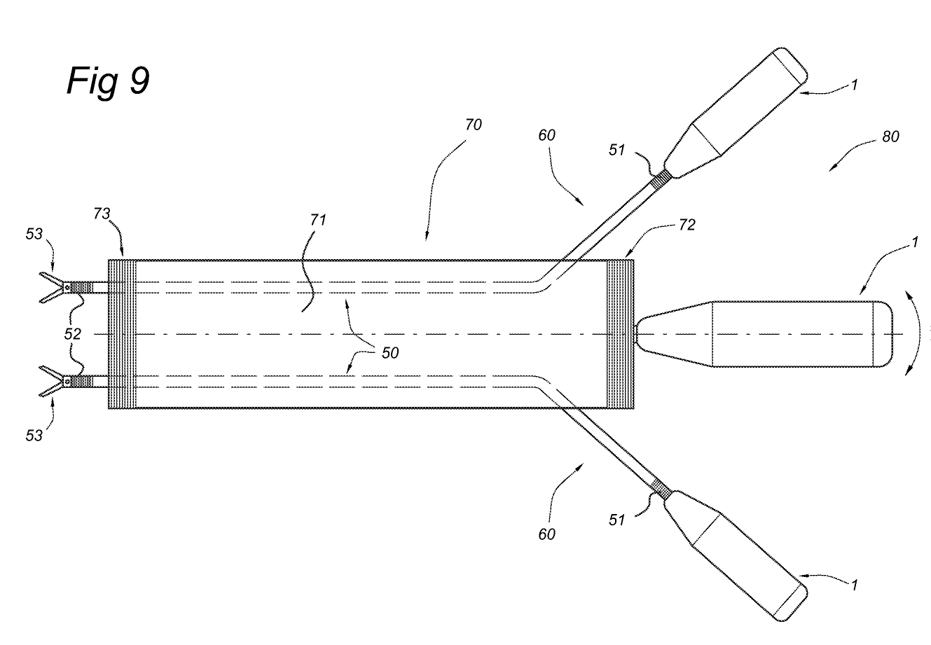

FIG. 9 shows a side view of an assembly 80 comprising two endoscopic instruments 60 that are guided through a single endoscope 70. The endoscopic instruments 60 and the endoscope 70 are constructed and arranged for being controlled by handles 1 according to the present invention. The endoscope 70 comprises a tube like element 71 having proximal 72 and distal 73 ends that comprise flexible portions that are constructed and arranged for steering the tube like element 71 of the endoscope 70 by moving the handle 1 in any direction.

FIG. 9 shows that the slim and compact pen-like construction of the handles 1 enables guiding two endoscopic instruments comprising such handles 1 side by side through the tube like element 71 of a single endoscope 70. As a result, it might no longer be necessary to make two incisions at sufficient distance from each other in certain applications. Consequently, an assembly 80 according to the present invention might actually enable minimal invasive interventions.

It will thus be seen that the invention efficiently attains the objects set forth above, among those made apparent from the preceding description. Since certain changes can be made in the above constructions without departing from the scope of the invention, it is intended that all matter contained in the above description or shown in the accompanying drawings be interpreted as illustrative and not in a limiting sense.

It is also to be understood that the following claims are to cover all generic and specific features of the invention described herein, and all statements of scope of the invention which, as a matter of language, might be said to fall therebetween.

What is claimed is:

1. Handle (1) for controlling instruments, comprising a frame (2) with a center line (9), operating elements (4, 10, 24) that are connected to the frame (2), which operating elements (4, 10, 24) are movable between a first and a second position, an actuator element (6) that is controllable by the operating elements (4, 10, 24), which actuator element (6) is constructed and arranged for controlling an instrument (53) that is arranged at a distance from the handle (1), wherein the operating elements (4, 10, 24) are constructed and arranged for enabling a longitudinal displacement of the actuator element (6) upon a movement of the operating elements (4, 10, 24) between the first and the second position, wherein the operating elements (4, 10, 24) are arranged in a rotationally symmetric configuration that is concentric with the center line (9) of the frame (2), wherein the operating elements (4, 10, 24) are movable between the first and second positions by a displacement in a direction substantially perpendicular to the center line (9) of the frame (2).

2. Handle (1) according to claim 1, wherein the handle (1) comprises a resilient element (14, 18) constructed and arranged for interconnecting the operating elements (4, 10, 24).

3. Handle (1) according to claim 2, wherein the resilient element (14, 18) rests on the operating elements (4, 10, 24).

4. Handle (1) according to claim 1, wherein the handle (1) has a rotationally symmetric outer circumference when the operating elements (4, 10, 24) are in the first and second positions.

5. Handle (1) according to claim 4, wherein the outer circumference of the handle (1) remains substantially rotationally symmetric when the operating elements (4, 10, 24) are moved between the first and second positions.

6. Handle (1) according to claim 1, wherein the handle (1) comprises a first pretensioning element (15) that is constructed and arranged for prestressing the operating elements (4, 10, 24) towards the first position.

7. Handle (1) according to claim 1, wherein the handle (1) comprises a second pretensioning element (14, 18) that is constructed and arranged for prestressing the operating elements (4, 10, 24) towards the second position.

8. Handle (1) according to claim 1, wherein the handle (1) comprises a housing (3), wherein the frame (2) and the housing (3) are connected such that their positions remain stationary with respect to each other when the operating elements (4, 10, 24) are moved from the first to the second position.

9. Handle (1) according to claim 1, wherein the operating elements (4, 10) are pivotable around pivot points (5) between the first and second position, wherein the pivot points (5) are arranged in a rotationally symmetric configuration that is concentric with the center line (9) of the frame (2).

10. Handle (1) according to claim 9, wherein the handle (1) comprises a element (5) constructed and arranged for interconnecting the pivot points.

11. Handle (1) according to claim 10, wherein element (5) rests on the operating elements (4, 10).

12. Handle (1) according to claim 9, wherein the operating elements (24) comprise first (29) and second (30) arms that are hingeably connected.

13. Handle (1) according to claim 9, wherein the operating elements (4, 10) are levers comprising first (7, 11) and second (8, 12) arms, wherein the first arms (7, 11) are positioned substantially radially with respect to the center line (9) of the frame (2) and the second arms (8, 12) are arranged substantially parallel to the center line (9) of the frame (2).

14. Handle (1) according to claim 13, wherein the handle (1) comprises a housing (3), wherein the frame (2) and the housing (3) are connected such that their positions remain stationary with respect to each other when the levers (4) are moved from the first to the second position.

15. Handle (1) according to claim 13, wherein the levers are divided into two groups, wherein a first group of levers (4) is arranged mirror wise with respect to a second group of levers (10).

16. Handle (1) according to claim 15, wherein the levers of the first (4) and second (10) groups are arranged in an interdigitated configuration.

17. Handle (1) according to claim 15, wherein the frame (2) and housing (3) are arranged such that they are movable with respect to each other when the levers (4, 10) are moved from the first to the second position.

18. Handle (1) according to claim 15, wherein each of the first (4) and second (10) groups of levers comprises at least one lever.

19. Handle (1) according to claim 1, wherein the handle (1) comprises a resilient sealing (14) constructed and arranged for sealing the operating elements (4, 10, 24) from an outside environment.

20. Handle (1) according to claim 19, wherein the resilient sealing (14) comprises a membrane comprising silicone or latex.

21. Endoscopic instrument (60) comprising a tube like element (50) with a proximal end (51) and a distal end (52), a tool (53) and a handle (1) according to claim 1, wherein the handle (1) and the tool (53) are arranged respectively at the proximal end (51) and the distal end (52) of the tube like element (50), wherein the tube like element (50) and the tool (53) are constructed and arranged for being operated by the handle (1).

22. Endoscopic instrument (60) according to claim 21, wherein the proximal (51) and distal (52) ends of the tube like element (50) comprise flexible portions that are constructed and arranged for steering the endoscopic instrument (60) by moving the handle (1) in any direction, wherein the tube like element (50) comprises longitudinal elements that are constructed and arranged for transferring a displacement of the proximal end (51) to the distal end (52) resulting in a change in orientation thereof, wherein the distal end (52) comprises at least two independent flexible portions, wherein the proximal (51) and distal (52) ends comprise a corresponding number of flexible portions, wherein each flexible portion at the proximal end (51) is connected by means of its own set of longitudinal elements to a flexible portion at the distal end (52).

23. Endoscopic instrument (60) according to claim 21, wherein the handle (1) comprises a frame (2) with a center line (9), operating elements (4, 10, 24) that are connected to the frame (2), which operating elements (4, 10, 24) are movable between a first and a second position, an actuator element (6) that is controllable by the operating elements (4, 10, 24), which actuator element (6) is constructed and arranged for controlling an instrument (53) that is located at a distal end (52) of the endoscope (50), wherein the operating elements (4, 10, 24) are constructed and arranged for enabling a longitudinal displacement of the actuator element (6) upon a movement of the operating elements (4, 10, 24) between the first and the second position, wherein the operating elements (4, 10, 24) are arranged in a rotationally symmetric configuration that is concentric with the center line (9) of the frame (2), wherein the operating elements (4, 10, 24) are movable between the first and second positions by a displacement in a direction substantially perpendicular to the center line (9) of the frame (2).

24. Endoscopic instrument (60) according to claim 21, wherein the handle (1) comprises a resilient element (14, 18) constructed and arranged for interconnecting the operating elements (4, 10, 24).

25. Endoscopic instrument (60) according to claim 24, wherein the resilient elements (14, 18) rests on the operating elements (4, 10, 24).

26. Endoscopic instrument (60) according to claim 21, wherein the handle (1) has a rotationally symmetric outer circumference when the operating elements (4, 10, 24) are in the first and second positions.

27. Endoscopic instrument (60) according to claim 26, wherein the outer circumference of the handle (1) remains substantially rotationally symmetric when the operating elements (4, 10, 24) are moved between the first and second positions.

28. Endoscopic instrument (60) according to claim 21, wherein the handle (1) comprises a first pretensioning element (15) that is constructed and arranged for prestressing the operating elements (4, 10, 24) towards the first position.

29. Endoscopic instrument (60) according to claim 21, wherein the handle (1) comprises a second pretensioning element (14, 18) that is constructed and arranged for prestressing the operating elements (4, 10, 24) towards the second position.

30. Endoscopic instrument (60) according to claim 21, wherein the handle (1) comprises a housing (3), wherein the frame (2) and the housing (3) are connected such that their positions remain stationary with respect to each other when the operating elements (4, 10, 24) are moved from the first to the second position.

31. Endoscopic instrument (60) according to claim 21, wherein the operating elements (4, 10) are pivotable around pivot points between the first and second position, wherein the pivot points (5) are arranged in a rotationally symmetric configuration that is concentric with the center line (9) of the frame (2).

32. Endoscopic instrument (60) according to claim 31, wherein the handle (1) comprises an element (5) constructed and arranged for interconnecting the pivot points.

33. Endoscopic instrument (60) according to claim 32, wherein the element (5) rests on the operating elements (4, 10).

34. Endoscopic instrument (60) according to claim 31, wherein the operating elements (24) comprise first (29) and second (30) arms that are hingeably connected.

35. Endoscopic instrument (60) according to claim 31, wherein the operating elements (4, 10) are levers comprising first (7, 11) and second (8, 12) arms, wherein the first arms (7, 11) are positioned substantially radially with respect to the center line (9) of the frame (2) and the second arms (8, 12) are arranged substantially parallel to the center line (9) of the frame (2).

36. Endoscopic instrument (60) according to claim 35, wherein the handle (1) comprises a housing (3), wherein the frame (2) and the housing (3) are connected such that their positions remain stationary with respect to each other when the levers (4) are moved from the first to the second position.

37. Endoscopic instrument (60) according to claim 35, wherein the levers are divided into two groups, wherein a first group of levers (4) is arranged mirror wise with respect to a second group of levers (10).

38. Endoscopic instrument (60) according to claim 37, wherein the levers of the first (4) and second (10) groups are arranged in an interdigitated configuration.

39. Endoscopic instrument (60) according to claim 37, wherein the frame (2) and housing (3) are moveably arranged such that they are movable with respect to each other when the levers (4, 10) are moved from the first to the second position.

40. Endoscopic instrument (60) according to claim 37, wherein each of the first (4) and second (10) groups of levers comprises at least one lever.

41. Endoscopic instrument (60) according to claim 21, wherein the handle (1) comprises a resilient sealing (14) constructed and arranged for sealing the operating elements (4, 10, 24) from an outside environment.

42. Endoscopic instrument (60) according to claim 41, wherein the resilient sealing (14) comprises a membrane comprising silicone or latex.

43. Assembly (80) comprising an endoscopic instrument (60) according to claim 21 and an endoscope (70) that is constructed and arranged for being controlled by the handle (1).

44. Assembly (80) according to claim 43, wherein the endoscope (70) comprises a tube like element (71) having a proximal end (72) and a distal end (73), wherein the proximal end (72) is connected to the handle (1), wherein the proximal (72) and distal (73) ends comprise flexible portions that are constructed and arranged for steering the endoscope (70) by moving the handle (1) in any direction, wherein the tube like element (71) comprises longitudinal elements that are constructed and arranged for transferring a displacement of the proximal end (72) to the distal end (73) resulting in a change in orientation thereof, wherein the distal end (73) comprises at least two independent flexible portions, wherein the proximal (72) and distal (73) ends comprise a corresponding number of flexible portions, wherein each flexible portion at the proximal end (72) is connected by means of its own set of longitudinal elements to a flexible portion at the distal end (73).
